(19) United States
(12) Patent Application Publication
(10) Pub. No.: US 2026/0120816 A1
(43) Pub. Date: Apr. 30, 2026

(54) PREDICTION METHOD FOR PROTOPORPHYRINOGEN OXIDASE (PPO) INHIBITOR

(71) Applicant: Pan Advanced Business Research, LLC, Kanagawa (JP)

(72) Inventor: Hirokazu OSABE, Yugawara-machi (JP)

(73) Assignee: Pan Advanced Business Research, LLC, Kanagawa (JP)

(21) Appl. No.: 19/488,745

(22) PCT Filed: Jun. 9, 2025
(86) PCT No.: PCT/JP2025/020862

§ 371 Date: Nov. 28, 2025

(30) Foreign Application Priority Data
Apr. 8, 2024 (JP) 2024-070084

(51) Int. Cl. G16C 20/30; G06F 17/18

(52) U.S. Cl. CPC G16C 20/30; G06F 17/18

(57) ABSTRACT

The present invention provides a technology that objectively and accurately predicts a degree of a PPO inhibitor using LUMO distribution as a “molecular descriptor” and a technology for selecting a PPO inhibitor based on the predicted degree of the PPO inhibitor. Provided are to predict PPO inhibitory activity based on a correlation between LUMO distribution and PPO inhibitory activity for each compound; to predict a degree of PPO inhibitory activity based on a correlation between a variation difference in PPO inhibitory activity and the degree of the PPO inhibitory activity between different plant species; and a method for selecting a PPO inhibitor, using the predicted PPO inhibitory activity as an index.

### TECHNICAL FIELD

The present invention relates to a prediction method for a PPO inhibitor, more specifically, a prediction method for a PPO inhibitor, using LUMO (Lowest Unoccupied Molecular Orbital) distribution as a “molecular descriptor,” and a method for selecting a PPO inhibitor, using the prediction method.

### BACKGROUND ART

In recent years, the identification of promising lead compounds, further optimization of the lead compounds, and eventual marketing as new herbicides require enormous financial costs in herbicide research and development, from the perspectives of excellent herbicidal activity, reduction of human toxicity and crop phytotoxicity, and ensuring safety for the natural environment.

As a technique for predicting the herbicidal activity of unmeasured compounds, a technique is used in which in silico methods are applied to statistically process the measurement data of numerous previously measured compounds (reference compounds) and the structural information of these compounds, and to predict the biological activity of the unmeasured compounds based on the degree of the structural similarity between the reference compounds and the unmeasured compounds. This inductive method is based on the hypothesis that “if compounds have similar structures, they should also have similar biological activity, thus it is possible to predict biological activity by indicating the structural ‘closeness’ of the compounds.” For the structural information of compounds, numerical “Molecular descriptors” are used to make compounds' structural features, physicochemical properties, or the like (such as hydrophobicity and steric parameters) more computer-friendly. This technique is based on the Hansh-Fujita method (Non Patent Literature 1), which serves as the starting point for various techniques that quantitatively handle the relationship between chemical structure and physiological activity. The molecular descriptors include hydrophobicity (Log P, π) and steric parameters (MR, STERIMOL, or the like).

The aforementioned inductive method can utilize various measurements, such as cell-based and enzyme-based measurements and animal testing, for herbicide research, from databases like PubChem (https://pubchem.ncbi.nlm.nih.gov/). However, since various measurements for researching the correlation between the structure of necessary compounds and the herbicidal activity are quantitatively databased for similar compounds, it is extremely difficult to say that they can be readily prepared in a short period of time. Thus, objective prediction approaches are still insufficient, and the development of such technology has been strongly desired.

Therefore, to efficiently and effectively research and develop candidate herbicidal compounds, it is necessary to create a significant “molecular descriptor” that predicts structural and electronic complementarity for each pair of a compound that is an inhibitor and a target enzyme such as PPO, whose three-dimensional structure is known, and use it in quantitative prediction methods for enzyme inhibitors.

Plant PPO inhibitors require light for herbicidal activity and are also called “light-dependent herbicides.” Research on the mechanism of action of “light-dependent herbicides” has been conducted for over half a century (Non Patent Literature 2), making it an inhibitor with a long history. Subsequent studies on enzyme levels revealed the following: plant PPO is an enzyme in the pathway synthesizing porphyrins from 5-Aminolevulinic acid (ALA) and is located within plant chloroplasts, and when the plant PPO is inhibited, protoporphyrinogen IX leaks from the chloroplasts into the cytoplasm, where it undergoes auto-oxidation to become protoporphyrin IX and accumulates within the cell (Non Patent Literature 3); protoporphyrin IX generates reactive oxygen species in the presence of light and oxygen, leading to peroxidative damage of biological membranes by light, causing plant cells to rapidly die, and the plant body to wither (Non Patent Literature 4).

Whether or not the plant PPO acts as the site of action is detected by a method using experimentally isolated plant PPO, and it is preferable to obtain enzyme material from chloroplasts not exposed to light (etioplasts).

For example, in the marketed herbicides, PPO inhibitory activity is measured as the pI50 of corn etioplasts (the logarithmic value of the inverse of the molar concentration of the compound that inhibits 50% of the PPO enzyme activity). In addition, there have been reported examples of herbicides including those represented by the following formulas, i.e., phenylpyrazole-based herbicide Pyraflufen Ethyl (Ethyl 2-chloro-5-(4-chloro-5-difluoromethoxy-1-methylpyrazol-3-yl)-4-fluorophenoxyacetate), diphenyl ether-based herbicide Oxyfluorofen (2-chloro-4-(trifluoromethyl)phenyl(3-ethoxy-4-nitrophenyl)ether), and cyclic imide-based herbicide Chlorophthalim (N-(4-chlorophenyl)-1-cyclohexene-1,2-dicarboximide) (Non Patent Literature 5).

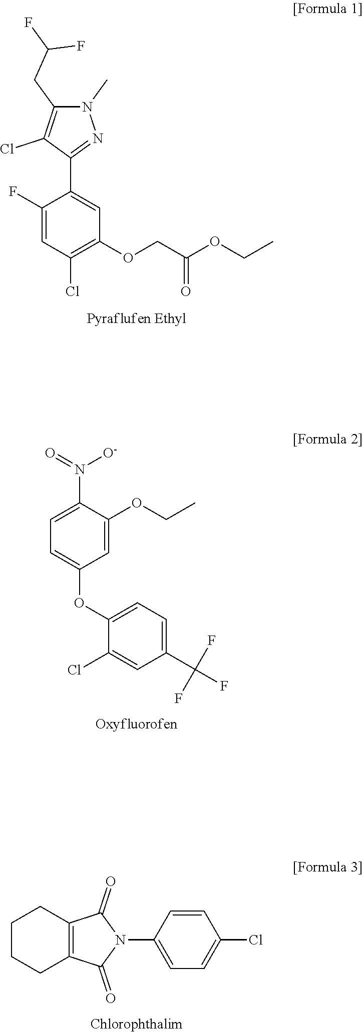

Other examples of pI50 using corn etioplasts include examples of various cyclic imide-based compounds containing Chlorophthalim (Non Patent Literature 6).

In other plant species, examples of pI50 using barley etioplast have been reported with various diphenyl ether-based compounds including Oxyfluorofen (Non Patent Literature 7).

On the other hand, a pyrazole-based compound B (1-(5-hydroxy-1,3-dimethyl-1H-pyrazol-4-yl)ethanone) represented by the following structural formula has been reported as a control compound which exhibits no herbicidal activity at all (Non Patent Literature 8).

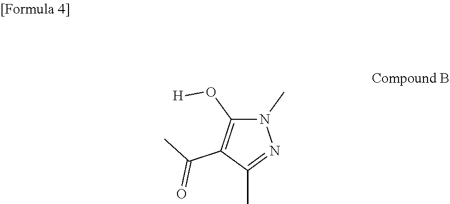

Regarding the crystal structure data of PPO derived from the tobacco plant (Nicotiana tabacum), the steric structure including phenylpyrazole-based PPO inhibitor A (4-bromo-3-(5′-carboxy-4′-chloro-2′-fluorophenyl)-1-methyl-5-trifluoromethylpyrazole) represented by the following structural formula has been reported (Non Patent Literature 9), and the crystal structure data can be obtained from the Protein Data Bank (PDB) with PDB ID 1SEZ.

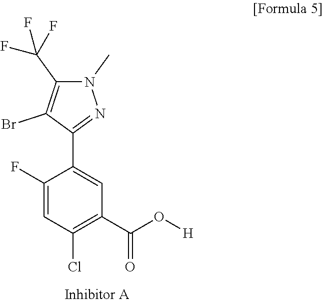 [SMILES: Cn1nc(-c2cc(C(=O)O)c(Cl)cc2F)c(Br)c1C(F)(F)F]

The PPO crystal structure data derived from Myxococcus xanthus, including a diphenyl ether-based PPO inhibitor Acifluorfen (5-[2-chloro-4-(trifluoromethyl) phenoxy]-2-nitrobenzoic acid) represented by the following structural formula (Non Patent Literature 10) and those derived from humans (Homo sapiens) (Non Patent Literature 11) can be obtained from the PDB with PDB IDs 12IVD and 3NKS, respectively.

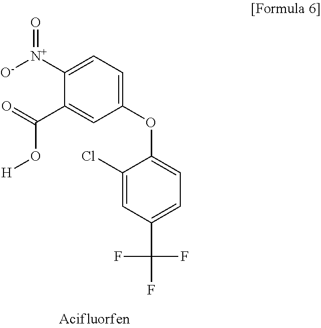 [SMILES: O=C(O)c1cc(Oc2ccc(C(F)(F)F)cc2Cl)ccc1[N+](=O)[O-]]

Firstly, to generate a “molecular descriptor” that quantifies the PPO enzyme inhibition reaction, the present inventor has focused on the oxidized flavin adenine dinucleotide (FAD), a coenzyme of PPO, represented by the following structural formula. In other words, the present inventor has hypothesized that oxidized FAD, which functions as an electrophilic electron acceptor under normal conditions, might have its electrophilicity inhibited by PPO inhibitors.

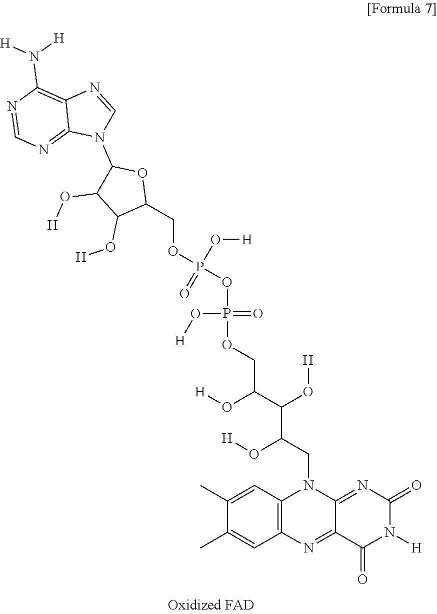 [SMILES: Cc1cc2nc3c(=O)[nH]c(=O)nc-3n(CC(O)C(O)C(O)COP(=O)(O)OP(=O)(O)OCC3OC(n4cnc5c(N)ncnc54)C(O)C3O)c2cc1C]

However, there have been no examples that details, based on the steric structures, how PPO controls the electron transfer reaction using oxidized FAD as an electron acceptor. Therefore, the present inventor has predicted the chemical reactions based on the interaction between the Highest Occupied Molecular Orbital (HOMO) of the electron donor molecule and the Lowest Unoccupied Molecular Orbital (LUMO) of the electron acceptor molecule using the Frontier orbital theory (Non Patent Literature 12). Specifically, the present inventor considered that a correlation with PPO inhibitory activity would be established if a “molecular descriptor” is set to quantify and indicate the change in the LUMO distribution of the oxidized FAD caused by the delocalization of electrons, which results from the mixing of electrons localized at the HOMO of the PPO inhibitor with the LUMO of the oxidized FAD molecule due to the interaction with the oxidized FAD.

It has not been known at all that the LUMO distribution of the two-molecule complex of the oxidized FAD and a PPO inhibitor at the PPO inhibitory activity site can be used as a “molecular descriptor” to accurately predict the degree of PPO inhibitor. Nor has it been known at all to provide a method for selecting a PPO inhibitor based on the degree of the PPO inhibitor predicted in such a manner.

### CITATION LIST

### Non Patent Literature

Non Patent Literature 1: C. Hansch and T. Fujita, J. Am. Chem. Soc., (1964) 86, 1616-1626
Non Patent Literature 2: S. Matsunaka, J. Agric. Food Chem, (1969) 17 (2), 171-175
Non Patent Literature 3: M. Matringe et al., The Biochemical Journal, (1989) 60, 231-235
Non Patent Literature 4: H. J. Lee et al., Plant Physiol., (1993) 102, 881-889
Non Patent Literature 5: S. Ishida et al., J. Pesticide Sci., (2000), 25, 18-23
Non Patent Literature 6: T. Fujita and A. Nakayama, Peroxidizing Herbicide, Eds. P. Boeger and K. Wakabayashi, Springer, Berlin Heidelberg, (1999), 91-139
Non Patent Literature 7: U. B. Nandihalli et al., Pesticide Biochemistry and Physiology, (1992), 43, 193-211
Non Patent Literature 8: M. Nakagawa et al., J. Pesticide. Sci., (1988) 13, 363-374
Non Patent Literature 9: M. Koch et al., The EMBO Journal, (2004), 23, 1720-1728
Non Patent Literature 10: H. R. Corradi et al., J BiolChem., (2006), 281 (50), 38625-38633
Non Patent Literature 11: X. Qin et al., FASEB J., (2011), 25, 653-664
Non Patent Literature 12: Kenichi Fukui, “Chemical reactions and electron orbitals,” Maruzen, 1976

### SUMMARY OF INVENTION

### Technical Problem

An object of the present invention is to provide a technology that objectively, accurately, and precisely predicts the degree of PPO inhibitors using LUMO distribution as a “molecular descriptor” and a technology for selecting a PPO inhibitor based on the predicted degree of a PPO inhibitor.

### Solution to Problem

Considering such circumstances, the present inventor has diligently conducted research and efforts seeking a technology that objectively, accurately, and precisely predicts the degree of PPO inhibitors. As a result, the present inventor has found that it is possible to objectively and accurately predict the degree of PPO inhibitors based on the correlation between the LUMO distribution and the degree of PPO inhibitors for each compound, and have completed the invention. That is, the present invention relates to the technologies described below.
(1) A prediction method for a PPO inhibitor, comprising predicting a degree of the PPO inhibitor using LUMO distribution as a “molecular descriptor.”
(2) The prediction method for a PPO inhibitor according to claim 1, wherein PPO inhibitory activity is predicted based on a correlation between the LUMO distribution and the PPO inhibitory activity for each compound.
(3) The prediction method for a PPO inhibitor according to claim 1 or 2, wherein the prediction of the PPO inhibitory activity based on the correlation is calculated in corn and barley using formulas below:

$$\begin{matrix}〈 Corn 〉 & \\ PPO inhibitory activity = 0.662 * LUMO distribution index + 5.795 ; & \end{matrix}$$

$$and$$

$$\begin{matrix}〈 Barley 〉 & \\ PPO inhibitory activity = 0.987 * LUMO distribution index + 2.363 . & \end{matrix}$$

(4) The prediction method for a PPO inhibitor according to claim 1, wherein a degree of PPO inhibitory activity is predicted based on a correlation between a variation difference in PPO inhibitory activity and the degree of PPO inhibitory activity between different plant species.
(5) A method for selecting a PPO inhibitor, comprising using the PPO inhibitory activity predicted by (1) to (4) as an index.
(6) A PPO inhibitor predicted or selected by the method according to one of (1) to (5).

### Advantageous Effects of Invention

According to the present invention, it is possible to provide a technology that objectively and accurately predicts the degree of PPO inhibitors.

### BRIEF DESCRIPTION OF DRAWINGS

FIG. 1 is a diagram illustrating the change in LUMO distribution of corn pI50.

FIG. 2 is a diagram illustrating the change in LUMO distribution of barley pI50.

FIG. 3 is a diagram illustrating a correlation between the variation difference in PPO inhibitory activity and the degree of PPO inhibitory activity between different plant species.

FIG. 4 is a diagram illustrating the LUMO of oxidized FAD alone.

FIG. 5 is a diagram illustrating the LUMO of an inhibitor A complex.

FIG. 6 is a diagram illustrating the LUMO of a compound B complex.

FIG. 7 is a diagram illustrating the LUMO of a Pyraflufen Ethyl complex.

FIG. 8 is a diagram illustrating the LUMO of an Acifluorfen complex.

FIG. 9 is a diagram illustrating the LUMO of an Oxyfluorofen complex.

### DESCRIPTION OF EMBODIMENTS

### (1) Prediction Method for PPO Inhibitor of Present Invention

The prediction method for a PPO inhibitor of the present invention is characterized by using LUMO distribution as a “molecular descriptor” to predict a degree of the PPO inhibitor. To perform the prediction, it is necessary to clarify the relationship between the degree of PPO inhibitors and the LUMO distribution, and create a regression equation or similar expression that indicates this relationship for use in the prediction. The details are described below.

The general inhibitory activity of PPO inhibitors is represented by pI50, and a larger value indicates stronger activity. The corn pI50 for Pyraflufen Ethyl is obtained from Non Patent Literature 5, for the cyclic imide-based compounds from Non Patent Literature 6, and for Chlorophthalim from Non Patent Literatures 5 and 6. The barley pI50 for the diphenyl ether-based compounds is obtained from Non Patent Literature 7. These pI50 are used for the analysis. Moreover, the corn pI50 for Oxyfluorofen is obtained from Non Patent Literature 5, and the barley pI50 for Oxyfluorofen is obtained from Non Patent Literature 7, for use in the analysis.

It is desirable that the LUMO of the conformation of the two-molecule complex of the oxidized FAD and an inhibitor to be predicted is calculated using the non-empirical molecular orbital method/density functional theory calculation program GAMESS, and that the calculation is performed with the 6-31G (2d, p) basis set using the B3LYP functional of the DFT (Density Functional Theory) method.

The method of representing the LUMO distribution as a “molecular descriptor” can be performed in accordance with the following qualitative standard method, where comparison to the LUMO distribution of oxidized FAD alone is performed, and ranking is performed according to the following criteria: 5=75% or more and up to 100% disappearance; 4=50% or more and less than 75% disappearance; 3=25% or more and less than 50% disappearance; 2=1% or more and less than 25% disappearance; and 1=no effect.

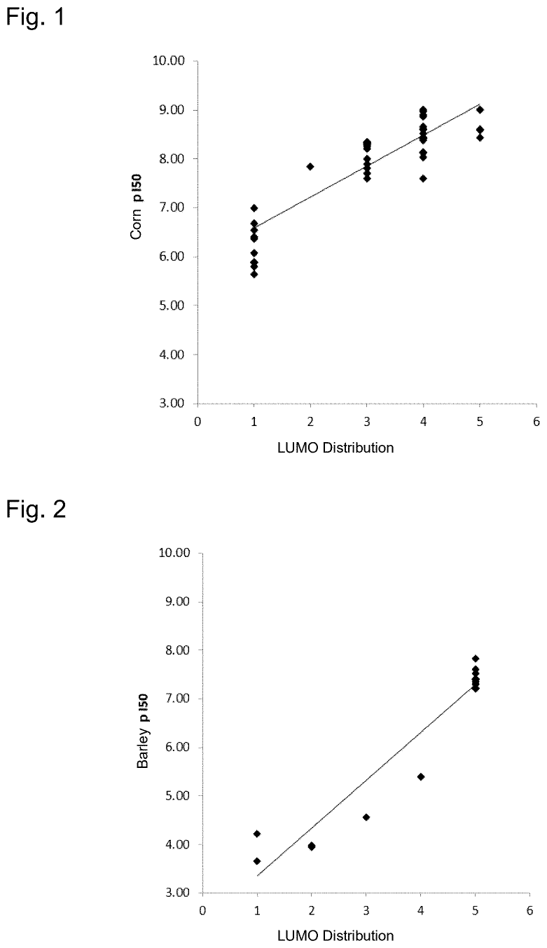

To clarify such relationships, a correlation analysis and regression analysis of corn PPO inhibitory activity and LUMO distribution have been conducted on the corn pI50 of Pyraflufen Ethyl and Oxyfluorofen (Non Patent Literature 5) and on the corn pI50 of 41 compounds including Chlorophthalim extracted from the cyclic imide-based compounds (Non Patent Literature 6). This shows a significant correlation (correlation coefficient=0.90) (see FIG. 1), and it is found that, by using the following regression equation calculated in this manner, the corn PPO inhibitory activity can be predicted with high accuracy from the LUMO distribution.

$$\begin{matrix}〈 Corn 〉 & \\ pI 50 = 0.662 * LUMO distribution + 5.795 & \end{matrix}$$

$$( Correlation coefficient = 0.9 )$$

Also, a correlation analysis and regression analysis of barley PPO inhibitory activity and LUMO distribution have been conducted by extracting 16 diphenyl ether-based compounds including Oxyfluorofen, and conducting correlation analysis and regression analysis on barley pI50 (Non Patent Literature 7) and LUMO distribution. This shows a significant correlation (correlation coefficient=0.96) (see FIG. 2), and it is found that, by using the following regression equation calculated in this manner, the PPO inhibitory activity can be predicted with high accuracy from the LUMO distribution.

$$\begin{matrix}〈 Barley 〉 & \\ pI 50 = 0.987 * LUMO distribution + 2.363 & \end{matrix}$$

$$( Correlation coefficient = 0.96 )$$

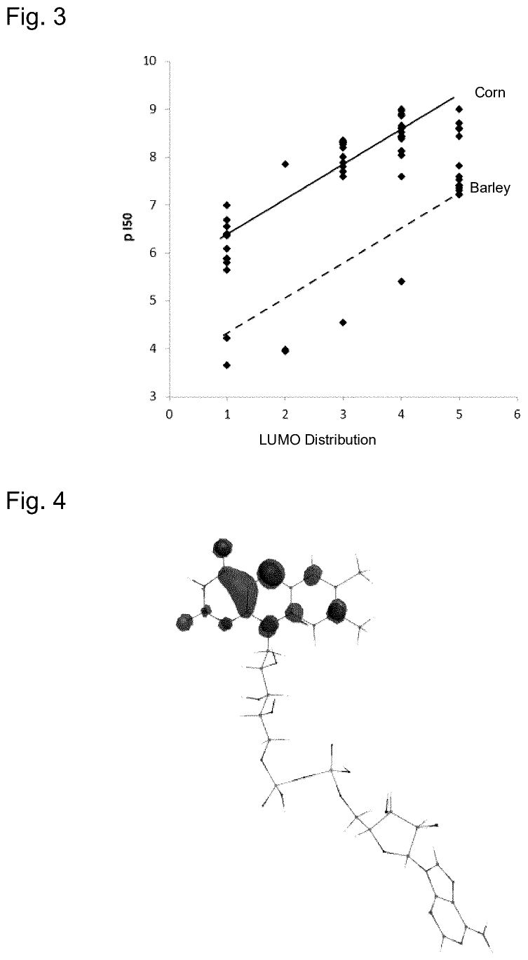

Based on the regression equation for the variation difference in pI50 between different plant species, namely corn and barley described above, and the LUMO distribution, an analysis is performed by adding a dummy variable (I[barley]) assigned a value of 1 for barley pI50. Similarly, this shows a significant correlation (see FIG. 3). It is found that, by using the following regression equation calculated in such a manner, the degree of PPO inhibitory activity between different plant species can be predicted with high accuracy.

$$pI 50 = 0.768 * LUMO distribution - 2.238 * I [ barley ] + 5.465$$

$$( Correlation coefficient = 0.94 )$$

The aforementioned correlation analysis, regression analysis, and the like have been performed using MS Office Excel. However, they can also be carried out using, for example, freeware or commercially available general-purpose multivariate analysis software.

In this way, it is understood that, by using the LUMO distribution as the “molecular descriptor,” the degree of PPO inhibitors can be predicted with high accuracy. However, optionally, other characteristic values related to the PPO inhibitors, such as hydrophobicity (Log P) and steric parameters (MR, STERIMOL, or the like), may be added as the “molecular descriptors.” In other words, just like the “molecular descriptor” of LUMO distribution, by incorporating these into the “molecular descriptors” to create a multiple regression analysis formula, they can contribute to improving the prediction accuracy of PPO inhibitory activity. Such a range also falls within the technical scope of the present invention.

### (2) Method for Selecting PPO Inhibitor of Present Invention

The method for selecting a PPO inhibitor of the present invention is characterized by predicting a PPO inhibitor based on the degree of PPO inhibitory activity predicted as described above. The ranks of the LUMO distribution are not particularly limited, but 4 or 5 ranks are preferred.

Hereinafter, the present invention will be described in further detail with reference to Examples, but it goes without saying that the present invention is not limited to only these Examples.

### EXAMPLES

### Example 1

### <Calculation of LUMO of Oxidized FAD Alone>

The PPO crystal structure data with ID 1SEZ was acquired from the PDB, only the conformation of the oxidized FAD was extracted from this conformation data, and hydrogen was added to the conformation using an open-source modeling software Molby. This was designated as the oxidized FAD alone. The GAMESS input file for this oxidized FAD alone was created using Molby. First, geometry optimization of all interatomic distances, bond angles, and dihedral angles was performed by GAMESS using a semi-empirical MO method calculation technique PM3. Afterwards, using the optimized structure, further geometry optimization was performed on all interatomic distances, bond angles, and dihedral angles using the 3-21G basis set with the Hartree-Fock (HF) method.

Using the optimized conformation, a single-point calculation was performed by the 6-31G (2d, p) basis set calculation using the B3LYP functional according to the DFT method to determine LUMO.

The calculation results are shown in FIG. 4, by calculating the LUMO of the oxidized FAD alone using an open-source modeling software Avogadro with Isosuraface Value set to 0.05. The figure shows the region where the LUMO is distributed on the isoalloxazine ring in the molecular structure, and simultaneously indicates that electrons are absent in the region where the LUMO is present, which implies that an electrophilic reaction may occur on the isoalloxazine ring due to the absence of electrons. This was considered as the LUMO of the oxidized FAD alone as an electron acceptor in normal PPO that is not inhibited by PPO inhibitors.

### Example 2

### <Calculation of LUMO of Inhibitor A Complex>

The PPO crystal structure data with the ID 1SEZ was acquired from the PDB as in Example 1. From this conformation data, only the conformation of the two-molecule complex of the oxidized FAD and an inhibitor A was extracted, and the extracted conformation was designated as an inhibitor A complex.

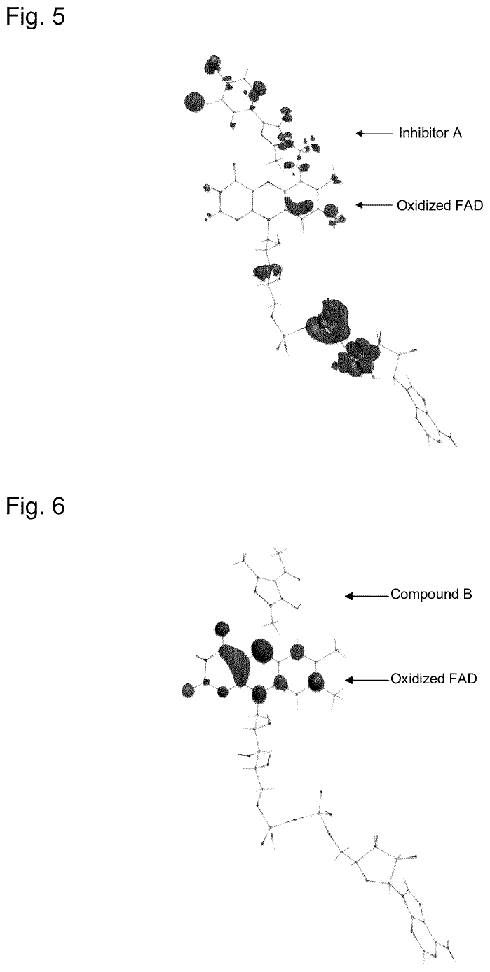

The LUMO of the inhibitor A complex was calculated as in Example 1, and is shown in FIG. 5. In the inhibitor A complex, the LUMO on the isoalloxazine ring, which was present in the oxidized FAD alone, almost disappeared. This indicates that the electrons that had been localized in the HOMO of the inhibitor A were mixed due to the interaction with the oxidized FAD, resulting in the delocalization of the electrons into the oxidized FAD molecule. This means the potential inhibition of the electrophilic reaction of the oxidized FAD.

### Example 3

### <Calculation of LUMO of Compound B Complex>

As a control compound with no herbicidal activity, a compound B complex consisting of a two-molecule complex of a compound B and the oxidized FAD was prepared by performing structure modification such as substituent changes of the inhibitor A in the inhibitor A complex of Example 2 using a molecular modeling software Winmostar 64-bit (FREE) v11.6.1.

LUMO was calculated as in Examples 1 and 2, and is shown in FIG. 6. In the compound B complex, the region where the LUMO is distributed on the isoalloxazine ring matched with the LUMO of the oxidized FAD alone in Example 1. It was considered that this is because the compound B does not alter the LUMO on the isoalloxazine ring of the oxidized FAD, and a normal electrophilic reaction similar to the oxidized FAD alone under normal conditions can occur, thus it does not cause PPO inhibitory activity.

### Example 4

### <Calculation of LUMO of Pyraflufen Ethyl Complex>

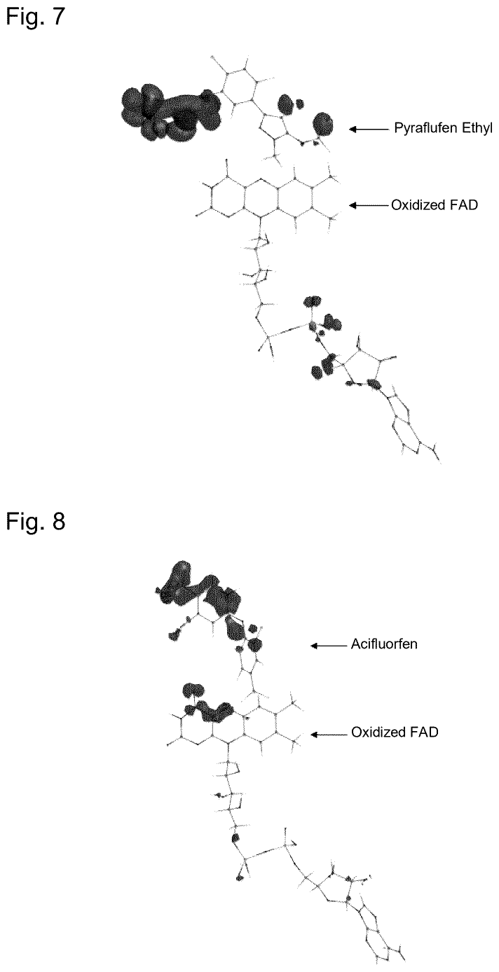

The LUMO of the two-molecule complex of Pyraflufen Ethyl and the oxidized FAD (Pyraflufen Ethyl complex) was calculated as in Examples 1 to 3, and is shown in FIG. 7.

The figure shows that, compared to the normal LUMO distribution shown in Examples 1 and 3, the LUMO on the isoalloxazine ring of the Pyraflufen Ethyl complex disappeared 100%, and the electrons that had been localized in the HOMO of the Pyraflufen Ethyl mixed due to interaction with the oxidized FAD, resulting in the delocalization of the electrons into the oxidized FAD molecule. This means the inhibition of the electrophilic reaction of the oxidized FAD.

### Example 5

### <Calculation Method for LUMO Distribution of Acifluorfen Complex>

PPO from Myxococcus xanthus with Acifluorfen was obtained from PDB ID 2IVD. From this conformation data, only the conformation of the two-molecule complex of the oxidized FAD and the PPO inhibitor Acifluorfen was extracted, and hydrogen was added using Molby. The conformation was designated as a 2IVD complex.

The inhibitor A complex obtained in Example 2 and the 2IVD complex were superimposed at the isoalloxazine rings of the oxidized FAD using the Winmostar, and the inhibitor A of the inhibitor A complex was replaced with Aciflyorfen of the 2IVD complex to prepare an Acifluorfen complex composed of two molecules: the oxidized FAD of the inhibitor A complex and Acifluorfen of the 2IVD complex. The LUMO of the Acifluorfen complex was calculated as in Examples 1 to 4, and is shown in FIG. 8.

The figure shows that, similarly to the inhibitor A complex in Example 2, the LUMO on the isoalloxazine ring, which was present in the oxidized FAD alone, almost disappeared in the Acifluorfen complex, and the electrons that had been localized in the HOMO of Acifluorfen mixed due to the interaction with the oxidized FAD, resulting in the delocalization of the electrons into the oxidized FAD molecule. This means the inhibiting of the electrophilic reaction of the oxidized FAD by the electron reaction with Acifluorfen.

### Example 6

### <Calculation of LUMO for Oxyfluorofen Complex>

An Oxyfluorofen complex was prepared by performing structure modification such as substituent changes of Acifluorfen of the Acifluorfen complex in Example 5 using the Winmostar.

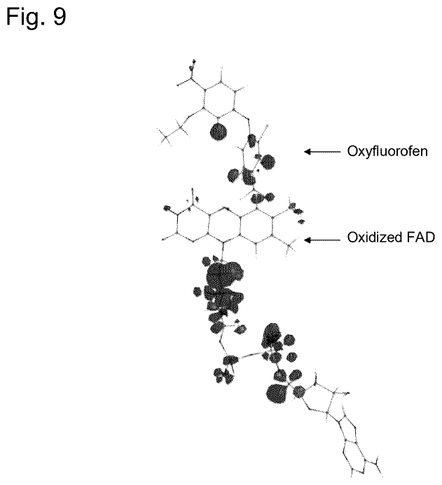

The LUMO of the Oxyfluorofen complex was calculated as in Example 5, and is shown in FIG. 9. The figure shows that, similarly to the Pyraflufen Ethyl complex in Example 5, the LUMO on the isoalloxazine ring disappeared 100%, and similarly to the inhibitor A, the electrons that had been localized in the HOMO of Oxyfluorofen mixed due to interaction with the oxidized FAD, resulting in the delocalization of the electrons into the oxidized FAD molecule. This means the inhibition of the oxidized FAD electrophilic reaction.

### Example 7

### <Calculation of LUMO Distribution in Corn Correlation Analysis>

In addition to the LUMOs of the Pyraflufen Ethyl complex of Example 4 and the Oxyfluorofen complex of Example 5, the LUMOs of the complexes of Chlorophthalim and the cyclic imide-based compounds Nos. 1 to 40, whose substituents are specified in the following general formula (I), and the oxidized FAD were each determined by the techniques of Examples 1 to 6. The LUMO distributions for each complex No. were ranked as follows and are listed in Table 1 below.

Compared to the LUMO distribution of the oxidized FAD alone, 5=75% or more and up to 100% disappearance; 4=50% or more and less than 75% disappearance; 3=25% or more and less than 50% disappearance; 2=1% or more and less than 25% disappearance and 1=no effect.

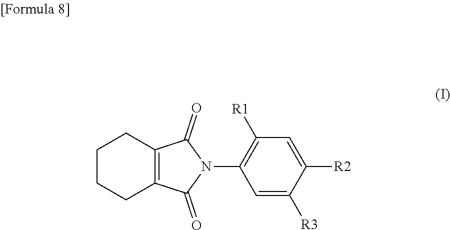

TABLE 1

| | | | | LUMO |
|---|---|---|---|---|
| No. | R1 | R2 | R3 | distribution |
| Pyraflufen | | | | 5 |
| ethyl | | | | |
| Oxyfluorfen | | | | 5 |
| Chlorophthalim | H | Cl | H | 4 |
| 1 | F | Cl | OCH(CH3)CCH | 4 |
| 2 | F | Cl | OCH2CCH | 4 |
| 3 | H | Cl | COOiC3H7 | 4 |
| 4 | F | Cl | COOiC3H7 | 4 |
| 5 | H | Cl | COOnC3H7 | 4 |
| 6 | F | Br | H | 5 |
| 7 | H | Cl | COOsC4H9 | 4 |
| 8 | F | Cl | OcycC5H11(3-CH3) | 4 |
| 9 | F | Cl | COOC2H5 | 5 |
| 10 | F | Cl | OCH3 | 4 |
| 11 | F | Cl | H | 5 |
| 12 | H | Cl | COOC2H5 | 4 |
| 13 | H | Cl | COOCH2CHCH2 | 4 |
| 14 | F | Cl | COOnC3H7 | 4 |
| 15 | F | Cl | COOallyl | 4 |
| 16 | F | Cl | COOiC4H9 | 3 |
| 17 | H | Cl | COOiC4H9 | 3 |
| 18 | F | Cl | COOsC4H9 | 3 |
| 19 | H | Cl | COOCH2COOCH3 | 3 |
| 20 | H | Cl | COSC2H5 | 3 |
| 21 | F | Cl | COOCH2CH2CN | 3 |
| 22 | H | Cl | COSCH3 | 4 |
| 23 | H | Cl | COOCH(CH3)COOCH3 | 4 |
| 24 | H | Cl | COOnC4H9 | 4 |
| 25 | H | Cl | COOCH2CCH | 3 |
| 26 | F | Cl | COOiC5H11 | 3 |
| 27 | H | Cl | COOtC4H9 | 2 |
| 28 | H | Cl | COOCH(CH3)COOCH3 | 3 |
| 29 | H | Cl | COOnC5H11 | 3 |
| 30 | H | Cl | COOnC8H17 | 3 |
| 31 | H | OCH3 | H | 1 |
| 32 | H | SCH3 | H | 1 |
| 33 | H | CF3 | H | 1 |
| 34 | H | NO2 | H | 1 |
| 35 | H | F | H | 1 |
| 36 | H | CH3 | H | 1 |
| 37 | CH3 | H | H | 1 |
| 38 | H | H | CH3 | 1 |
| 39 | H | H | H | 1 |
| 40 | F | H | H | 1 |

### Example 8

### <Calculation of LUMO Distribution in Barley Correlation Analysis>

In addition to the LUMO distributions of the Acifluorfen complex of Example 5 and the Oxyfluorofen complex of Example 6, the LUMO distributions of the complex of compounds Nos. 44 to 57, whose substituents are specified in the following general formula (II), and the oxidized FAD were calculated in the same manner as in Examples 5 to 7, and the LUMO distributions for each complex No. are listed in Table 2.

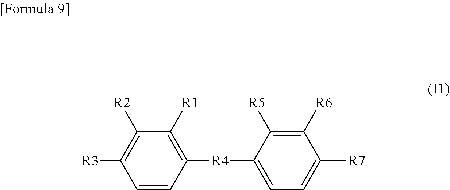

TABLE 2

| | | | | | | | | LUMO |
|---|---|---|---|---|---|---|---|---|
| No. | R1 | R2 | R3 | R4 | R5 | R6 | R7 | distribution |
| Acifluorfen | Cl | H | CF3 | O | H | COOH | NO2 | 4 |
| Oxyfluorofen | Cl | H | CF3 | O | H | OC2H5 | NO2 | 5 |
| 44 | Cl | H | CF3 | O | H | O-3-Furan | NO2 | 5 |
| 45 | Cl | H | CF3 | O | H | COOCH2COOH | NO2 | 5 |
| 46 | Cl | H | CF3 | O | H | COOCH3 | NO2 | 5 |
| 47 | Cl | H | CF3 | O | H | COOCH3 | Cl | 5 |
| 48 | Cl | H | CF3 | O | H | COOCH(CH3)COOC2H5 | NO2 | 5 |
| 49 | Cl | H | CF3 | S | H | COOCH3 | NO2 | 5 |
| 50 | Cl | H | Cl | O | H | H | NO2 | 5 |
| 51 | Cl | H | CF3 | O | H | H | NO2 | 5 |
| 52 | Cl | H | Cl | O | H | COOCH3 | NO2 | 5 |
| 53 | Cl | H | CF3 | SO | H | COOCH3 | NO2 | 3 |
| 54 | H | CH3 | H | O | H | H | NO2 | 1 |
| 55 | H | H | H | O | Cl | NH2 | NO2 | 2 |
| 56 | Cl | H | CF3 | SO2 | H | COOCH3 | NO2 | 2 |
| 57 | H | H | H | O | H | H | NO2 | 1 |

### INDUSTRIAL APPLICABILITY

According to the present invention, it is possible to provide a technology that objectively and accurately predicts the PPO inhibitory activity using LUMO distribution as a “molecular descriptor.” As a result, in herbicide studies, organic synthetic chemists can utilize this technology to predict the basic scaffold of PPO-inhibiting compound systems and to predict the selection of their substituents.

1. A prediction method for a PPO inhibitor, comprising:
creating a first regression equation based on a correlation between a LUMO distribution as a “molecular descriptor” and a PPO inhibitory activity for each compound having PPO inhibitory activity on one plant species, substituting the LUMO distribution as the “molecular descriptor” of a PPO inhibitor to be predicted into the first regression equation, and predicting a inhibitory activity of the PPO inhibitor to be predicted on the one plant species, wherein
the LUMO distribution as the “molecular descriptor” indicates, in a stepwise manner, a degree of disappearance of the LUMO distribution of a conformation of a two-molecule complex of oxidized FAD and the PPO inhibitor to be predicted, compared to the LUMO distribution of oxidized FAD alone.

2. The prediction method for a PPO inhibitor according to claim 1, wherein the prediction of the PPO inhibitory activity based on the first regression equation is calculated with corn as the one plant species using the formula below:
$$\begin{matrix}〈 Corn 〉 & \\ PPO inhibitory activity = 0.662 * LUMO distribution index + 5.795 . & \end{matrix}$$

3. The prediction method for a PPO inhibitor according to claim 1, wherein the prediction of the PPO inhibitory activity based on the the first regression equation is calculated in with barley as the one plant species using the formula below:
$$\begin{matrix}〈 Barley 〉 & \\ PPO inhibitory activity = 0.987 * LUMO distribution index + 2.363 . & \end{matrix}$$

4. A prediction method for a PPO inhibitor, comprising:
creating a second regression equation in which a correlation between PPO inhibitory activities and a LUMO distribution as a “molecular descriptor” between different plant species is indicated with a dummy variable that represents a variation difference in PPO inhibitory activities between the different plant species to be constant, and predicting a degree of difference of PPO inhibitory activities between the different plant species according to the variation difference in the created second regression equation, wherein
the LUMO distribution as the “molecular descriptor” indicates, in a stepwise manner, a degree of disappearance of the LUMO distribution of a conformation of a two-molecule complex of oxidized FAD and the PPO inhibitor, compared to the LUMO distribution of oxidized FAD alone.

5. (canceled)

6. (canceled)

7. The prediction method for a PPO inhibitor according to claim 4, wherein:
the plant species are corn and barley, and
the second regression equation is represented by the formula below:
wherein I[barley] is the dummy variable, and the I[barley] is 1 for barley
$$PPO inhibitory activity = 0.768 * LUMO distribution index - 2.238 * I [ barley ] + 5.465 .$$

8. A method for selecting a PPO inhibitor from candidate compounds having PPO inhibitory activity on one plant species, the method comprising:
selecting a PPO inhibitor from candidate compounds by comparing an index with a PPO inhibitory activity of an existing PPO inhibitor, wherein the index is a PPO inhibitory activity predicted using the first regression equation according to the prediction method for a PPO inhibitor according to claim 1 with the candidate compounds as the PPO inhibitor to be predicted.

9. The method for selecting a PPO inhibitor according to claim 8, wherein the existing PPO inhibitor exhibits 50% or more and up to 100% disappearance of the LUMO distribution of a conformation of a two-molecule complex of oxidized FAD and the existing PPO inhibitor compared to the LUMO distribution of oxidized FAD alone.

10. The method for selecting a PPO inhibitor according to claim 8, wherein the existing PPO inhibitor is any one PPO inhibitor selected from Pyraflufen Ethyl, Oxyfluorofen, Chlorophthalim, and Acifluorfen.

11. A method for selecting a PPO inhibitor from candidate compounds having PPO inhibitory activity on one plant species, the method comprising:
selecting a PPO inhibitor from the candidate compounds by comparing an index with a PPO inhibitory activity of the candidate compounds measured by an experimental method, wherein the index is a PPO inhibitory activity predicted using the first regression equation according to the prediction method for a PPO inhibitor according to claim 1 with the candidate compounds as the PPO inhibitor to be predicted.
